(19) United States
(12) Patent Application Publication
(10) Pub. No.: US 2008/0228255 A1
(43) Pub. Date: Sep. 18, 2008

(54) Positionable Stent-Graft Delivery System and Method

(75) Inventors: Matthew Rust, Santa Rosa, CA (US); Stephen Meier, San Francisco, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/685,251

(22) Filed: Mar. 13, 2007

(51) Int. Cl. A61F 2/06

(52) U.S. Cl. 623/1.11

(57) ABSTRACT

A positionable stent-graft delivery system includes a stent-graft, a tip capture mechanism radially constraining a proximal portion of an anchor stent ring of the stent-graft, and a positioning mechanism for positioning the tip capture mechanism. The positioning mechanism includes tensioner guides and cords. To position the tip capture mechanism, a cord is retracted through the respective tensioner guide, e.g., by the physician. Retraction of the cord, in turn, pulls the tip capture mechanism towards a distal end of the tensioner guide. In this manner, the stent-graft is readily repositioned.

### BACKGROUND OF THE INVENTION

1. Field of the Invention

This invention relates generally to medical devices and procedures, and more particularly to a method and system of deploying a stent-graft in a vascular system.

2. Description of Related Art

Prostheses for implantation in blood vessels or other similar organs of the living body are, in general, well known in the medical art. For example, prosthetic vascular grafts formed of biocompatible materials (e.g., Dacron or expanded, porous polytetrafluoroethylene (PTFE) tubing) have been employed to replace or bypass damaged or occluded natural blood vessels.

A graft material supported by a framework is known as a stent-graft or endoluminal graft. In general, the use of stent-grafts for treatment or isolation of vascular aneurysms and vessel walls which have been thinned or thickened by disease (endoluminal repair or exclusion) is well known.

Many stent-grafts, are “self-expanding”, i.e., inserted into the vascular system in a compressed or contracted state, and permitted to expand upon removal of a restraint. Self-expanding stent-grafts typically employ a wire or tube configured (e.g., bent or cut) to provide an outward radial force and employ a suitable elastic material such as stainless steel or Nitinol (nickel-titanium). Nitinol may additionally employ shape memory properties.

The self-expanding stent-graft is typically configured in a tubular shape of a slightly greater diameter than the diameter of the blood vessel in which the stent-graft is intended to be used. In general, rather than inserting in a traumatic and invasive manner, stents and stent-grafts are typically deployed through a less invasive intraluminal delivery, i.e., cutting through the skin to access a lumen or vasculature or percutaneously via successive dilatation, at a convenient (and less traumatic) entry point, and routing the stent-graft through the lumen to the site where the prosthesis is to be deployed.

Intraluminal deployment in one example is effected using a delivery catheter with coaxial inner tube, sometimes called the plunger, and sheath, arranged for relative axial movement. The stent-graft is compressed and disposed within the distal end of the sheath in front of the inner tube.

In other configurations balloon expandable stent grafts may have balloon expandable stents configured with a graft crimped on the outside of a delivery balloon which can be inflated by pressurizing a balloon inflation lumen of the catheter. There configurations do not require an outside sheath on the delivery catheter.

The catheter is then maneuvered, typically routed though a lumen (e.g., vessel), until the end of the catheter (and the stent-graft) is positioned in the vicinity of the intended treatment site. The inner tube is then held stationary while the sheath of the delivery catheter is withdrawn. For a self expanding configuration the inner tube prevents the stent-graft from moving back as the sheath is withdrawn.

As the sheath is withdrawn, the stent-graft is gradually exposed from a proximal end to a distal end of the stent-graft, the exposed portion of the stent-graft radially expands so that at least a portion of the expanded portion is in substantially conforming surface contact with a portion of the interior of the lumen, e.g., blood vessel wall.

One of the goals in placing the stent-graft, for example, to bypass an aneurysm in the abdominal aorta, is to place the proximal end of the graft material of the stent-graft as close to the top of the neck of the aneurysm as possible. More particularly, the proximal end of the graft material of the stent-graft should be placed as close to the renal arteries as possible without blocking the renal arteries to effectively bypass an aneurysm in the abdominal aorta.

In straight anatomies, placement of the stent-graft is relatively straightforward. However, in complex anatomies, e.g., in the case where the abdominal aorta curves dramatically from the renal arteries, placement of the stent-graft becomes less than ideal.

More particularly, in complex anatomies, the stent-graft is often deployed at an angle relative to a hypothetical square cylindrical surface section that is considered the neck of the aneurysm. This angular placement of the proximal end of the graft material of the stent-graft results in only one side of the graft material being placed right at the top of the neck and leaves a portion of the top of the neck of the aneurysm exposed (uncovered by the graft material). Accordingly, the sealing area (the contact area between the stent graft and the wall of the vessel (top neck of the aneurysm) between the graft material of the stent-graft and the neck of the aneurysm is reduced thus reducing the effectiveness of the seal and fixation between the graft material and the neck of aneurysm.

The proximal end of the stent-graft is considered to be the end closest to the heart whereas the distal end is the end furthest away from the heart during deployment and use. In contrast and of note, the distal end of the catheter is usually identified as the end that is farthest from the operator (handle) while the proximal end of the catheter is the end nearest the operator (handle). For purposes of clarity of discussion, as used herein (for a normal femoral approach), the distal end of the catheter is the end that is farthest from the operator (the end furthest from the handle) while the distal end of the stent-graft is the end nearest the operator (the end nearest the handle), i.e., the distal end of the catheter and the proximal end of the stent-graft are the ends furthest from the handle while the proximal end of the catheter and the distal end of the stent-graft are the ends nearest the handle. However, those of skill in the art will understand that depending upon the access location, the stent-graft and delivery system description may be consistent or opposite in actual usage.

### SUMMARY OF THE INVENTION

A positionable stent-graft delivery system (whether self expanding or balloon expandable) includes a stent-graft, a tip capture mechanism radially constraining a proximal anchor stent ring of the stent-graft, and a positioning mechanism for positioning the tip capture mechanism. The positioning mechanism includes tensioner guides and cords.

To position the tip capture mechanism, a cord is retracted through the respective tensioner guide, e.g., by the physician. Retraction of the cord, in turn, pulls the tip capture mechanism towards a distal end of the tensioner guide.

In this manner, the stent-graft is readily repositioned. For example, the stent-graft is repositioned to place the proximal end of a graft material of the stent-graft at the top of an aneurysmal neck to provide a maximum sealing area between the graft material and the aneurysmal neck.

An embodiment according to the invention includes a method of deploying a stent-graft with a positionable stent-graft delivery system including the steps of: radially constraining a proximal anchor stent ring of the stent-graft with a tip capture mechanism of the positionable stent-graft delivery system; radially constraining a graft material of the stent-graft with a primary sheath of the positionable stent-graft delivery system; partially retracting the primary sheath to expose a portion of the stent-graft; and retracting a cord through a tensioner guide of the positionable stent-graft delivery system to move the tip capture mechanism and reposition the proximal anchor stent ring and may further include the step of releasing the proximal anchor stent ring from the tip capture mechanism.

These and other features according to the present invention will be more readily apparent from the detailed description set forth below taken in conjunction with the accompanying drawings.

### BRIEF DESCRIPTION OF THE DRAWINGS

FIG. 1 is a partial cross-sectional view of a positionable stent-graft delivery system in accordance with one embodiment;

FIG. 2 is a cross-sectional view of the positionable stent-graft delivery system of FIG. 1 along the line II-II;

FIG. 3 is a schematicized perspective view of the positionable stent-graft delivery system of FIGS. 1 and 2;

FIG. 4 is a partial cross-sectional view of the positionable stent-graft delivery system of FIG. 1 after positioning;

FIG. 5 is a schematic view of a handle of the positionable stent-graft delivery system of FIG. 1;

FIG. 6 is a partial cross-sectional view of a positionable stent-graft delivery system located within a diseased vessel in accordance with one embodiment;

FIG. 7 is a partial cross-sectional view of the positionable stent-graft delivery system within the diseased vessel of FIG. 6 at a later stage during deployment of a stent-graft of the positionable stent-graft delivery system;

FIG. 8 is a partial cross-sectional view of the positionable stent-graft delivery system within the diseased vessel of FIG. 7 at a later stage during deployment of the stent-graft; and

FIG. 9 is a partial cross-sectional view of the stent graft of the positionable stent-graft delivery system finally deployed within the diseased vessel of FIG. 8.

In the following description, the same or similar elements are labeled with the same or similar reference numbers.

### DETAILED DESCRIPTION

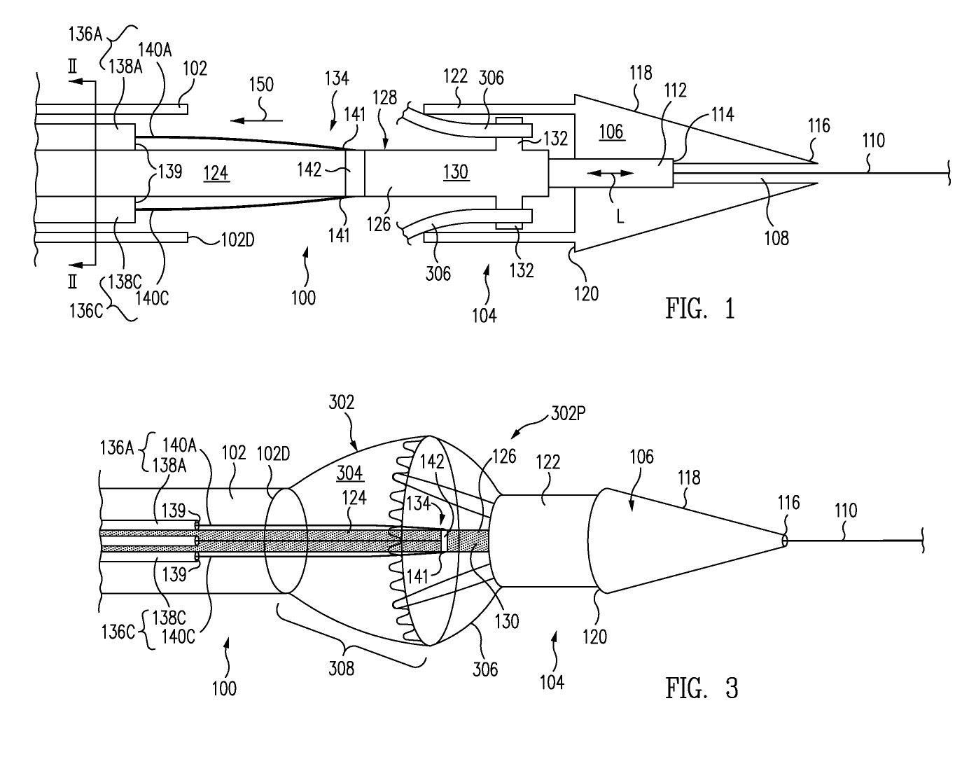

In accordance with one embodiment, referring to FIGS. 1, 2, and 3, a positionable stent-graft delivery system 100 includes a stent-graft 302, a tip capture mechanism 104 radially constraining a proximal anchor stent ring 306 of stent-graft 302, and a positioning mechanism 134 for positioning tip capture mechanism 104. Positioning mechanism 134 includes tensioner guides 138A, 138B, 138C, 138D, collectively tensioner guides 138, and cords 140A, 140B, 140C, 140D, collectively cords 140.

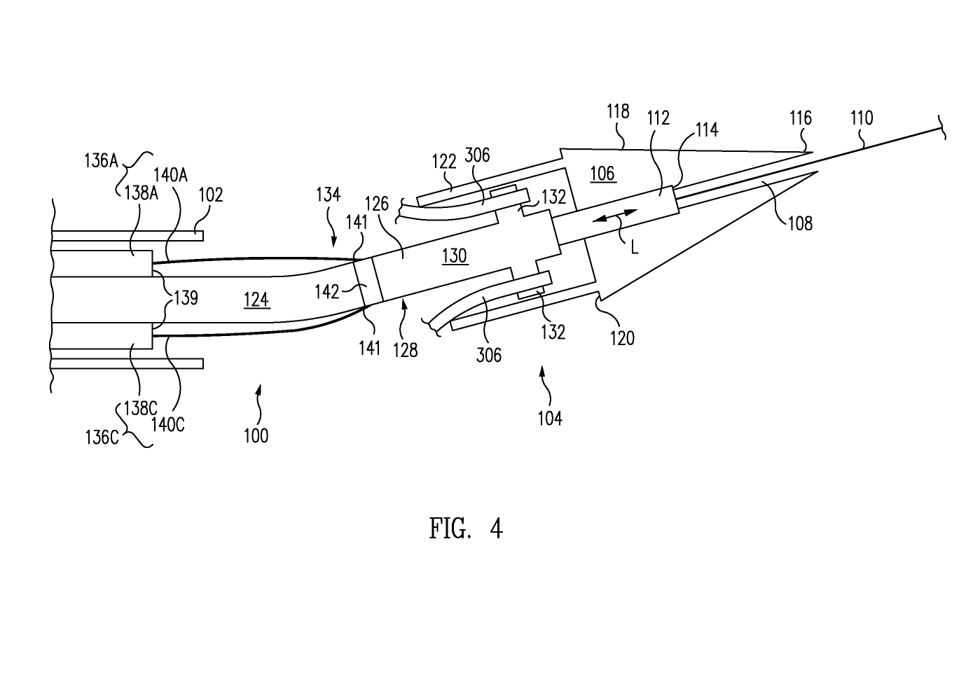

Referring now to FIGS. 1 and 4 together, to position tip capture mechanism 104 (as more fully described in U.S. Patent Application 2004/0093063 A1 (incorporated in its entirety by this reference herein)), cord 140A is retracted in the direction of arrow 150 of FIG. 1 through tensioner guide 138A, e.g., by the physician. Retraction of cord 140A, in turn, pulls tip capture mechanism 104 towards distal end 139 of tensioner guide 138A.

In this manner, the end of the stent-graft 302 is readily repositioned. For example, stent-graft 302 is repositioned to align the proximal end of a graft material 304 of stent-graft 302 more closely with the top end of an aneurysmal neck to provide a maximum sealing area between graft material 304 and the aneurysmal neck.

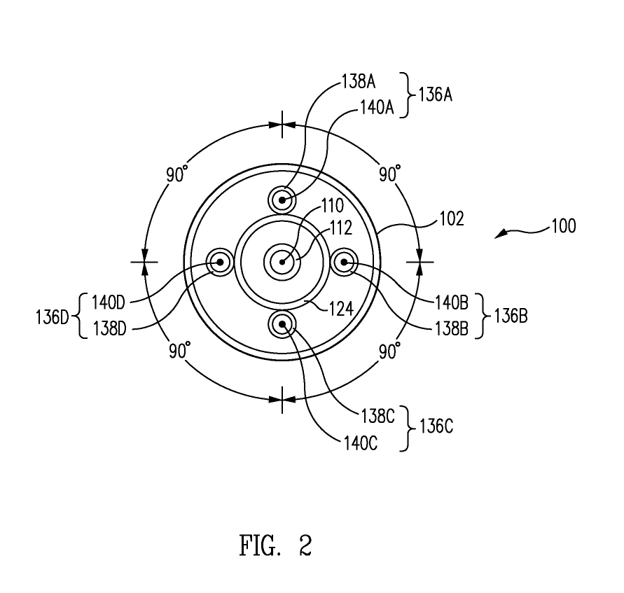

FIG. 1 is a partial cross-sectional view of a positionable stent-graft delivery system 100 in accordance with one embodiment. FIG. 2 is a cross-sectional view of positionable stent-graft delivery system 100 of FIG. 1 along the line II-II. FIG. 3 is a schematicized perspective view of positionable stent-graft delivery system 100 of FIGS. 1 and 2. For purposes of clarity of illustration, only a portion of a proximal anchor stent ring 306 of a stent-graft 302 is illustrated in FIG. 1. Further, in FIG. 3, the graft material 304 of stent-graft 302 and a primary sheath 102 are illustrated as being transparent to allow the visualization of the features therein for clarity of presentation. However, it is to be understood that in other examples, graft material 304 and/or primary sheath 102 are opaque.

Referring now to FIGS. 1, 2 and 3 together, positionable stent-graft delivery system 100, sometimes called a positionable prosthesis delivery system, includes a tip capture mechanism 104. Generally, graft material 304 of stent-graft 302 is radially constrained by primary sheath 102 and the proximal portion of proximal anchor stent ring 306 of stent-graft 302 is radially constrained by tip capture mechanism 104 allowing sequential and independent deployment of graft material 304 and proximal anchor stent ring 306 of stent-graft 302.

A tip capture mechanism similar to tip capture mechanism 104 is also described in Mitchell et al., U.S. patent application Ser. No. 11/559,754, filed on Nov. 14, 2006, entitled “DELIVERY SYSTEM FOR STENT-GRAFT WITH ANCHORING PINS”, which is herein incorporated by reference in its entirety. A brief description of tip capture mechanism 104 is set forth below. However, in light of this disclosure, those of skill in the art will understand that other tip capture mechanisms can be used in other examples.

Positionable stent-graft delivery system 100 includes a tapered tip 106 that is flexible and able to provide trackability in tight and tortuous vessels. Tapered tip 106 includes a guidewire lumen 108 therein for allowing passage of a guidewire 110 through tapered tip 106. Other tip shapes such as bullet-shaped tips could also be used.

An inner tube 112 defines a lumen, e.g., a guide wire lumen, therein. A distal end 114 of inner tube 112 is located within and secured to tapered tip 106, i.e., tapered tip 106 is mounted on inner tube 112. As shown in FIG. 1, the lumen of inner tube 112 is in geometric alignment (fluid communication) with guidewire lumen 108 of tapered tip 106 such that guide wire 110 is passed through inner tube 112 and out distal end 114, through guidewire lumen 108 of tapered tip 106, and out a distal end 116 of tapered tip 106.

Tapered tip 106 includes a tapered outer surface 118 that gradually increases in diameter. More particularly, tapered outer surface 118 has a minimum diameter at distal end 116 and gradually increases in diameter proximally, i.e., in the direction of the operator (or handle of positionable stent-graft delivery system 100), from distal end 116.

Tapered outer surface 118 extends proximally to a primary sheath abutment surface (shoulder) 120 of tapered tip 106. Primary sheath abutment surface 120 is an annular ring surface perpendicular to a longitudinal axis L of positionable stent-graft delivery system 100.

Tapered tip 106 further includes a (tip) sleeve 122 which is a hollow cylindrical tube extending proximally and longitudinally from primary sheath abutment surface 120.

Positionable stent-graft delivery system 100 further includes an outer tube 124 having a spindle 126 located at and fixed to a distal end 128 of outer tube 124. Spindle 126 includes a spindle body 130 having a cylindrical outer surface and a plurality of spindle pins 132 protruding radially outward from spindle body 130.

As illustrated in FIG. 1, spindle 126 is configured to slip inside of sleeve 122 such that spindle pins 132 are directly adjacent to, or contact, sleeve 122. Spindle pins 132 extend from spindle body 130 radially outward toward and to sleeve 122.

Further, the proximal crowns of the proximal anchor stent ring 306 of stent-graft 302 are radially constrained and held in position around spindle pins 132 and in the annular space between spindle body 130 and sleeve 122 as illustrated in FIG. 1. The distal crowns of the proximal anchor stent ring 306 are connected to the proximal end 302P of stent-graft 302.

Inner tube 112 is within and extends through outer tube 124 and spindle 126. Inner tube 112 and thus tapered tip 106 is moved (advanced) along longitudinal axis L (longitudinally moved) relative to outer tube 124 and thus spindle 126 to release proximal anchor stent ring 306 of stent-graft 302. More particularly, tapered tip 106 is moved such that sleeve 122 uncovers spindle pins 132 thus allowing proximal anchor stent ring 306 to be release. The term “stent-graft” used herein should be understood to include stent-grafts and other forms of endoprosthesis.

Primary sheath 102 is a hollow tube and defines a lumen therein through which outer tube 124 and inner tube 112 extend. Primary sheath 102 includes a distal end 102D.

Prior to retraction of primary sheath 102, distal end 102D is adjacent to or in abutting contact with primary sheath abutment surface 120 of tapered tip 106. Distal end 102D fits snugly around sleeve 122 when primary sheath 102 is in its un-retracted pre-deployment position.

In the view of FIGS. 1 and 3, primary sheath 102 is partially retracted such that distal end 102D is spaced apart from tapered tip 106. Further, due to the retraction of primary sheath 102, a proximal portion 308 of stent-graft 302 is exposed and partially deployed. Proximal portion 308 is a portion of stent-graft 302, i.e., up to the proximal edge of the graft material and generally distal to proximal anchor stent ring 306 but proximal to the remaining portion of stent-graft 302.

As proximal portion 308 is only partially deployed and the proximal crowns of proximal anchor stent ring 306 remains radially constrained and un-deployed, stent-graft 302 can be repositioned in the event that the initial deployment position of stent-graft 302 is less than desirable.

To facilitate positioning (or repositioning) of tip capture mechanism 104 and thus stent-graft 302, positionable stent-graft delivery system 100 includes a positioning mechanism 134. Positioning mechanism 134 includes a plurality of tensioners 136. More particularly, positioning mechanism 134 includes four tensioners 136A, 136B, 136C, 136D, collectively tensioners 136, radially spaced equally from one another.

As shown in FIG. 2, in accordance with this example, each tensioner 136 is radially oriented 90 degrees from the immediately adjacent tensioner 136. To illustrate, tensioner 136B, e.g., a first tensioner, is positioned along a radius radially oriented 90 degrees from a radius associated with tensioner 136A, e.g., a second tensioner, and in the opposite direction of rotation is radially oriented 90 degrees from tensioner 136C, e.g., a third tensioner. As used herein, a radial position is a particular angular position along an imaginary circle lying in a plane perpendicular to longitudinal axis L and having longitudinal axis L at the center of the circle.

Each tensioner 136 includes a tensioner guide 138 and a cord 140. More particularly, tensioners 136A, 136B, 136C, 136D include tensioner guides 138A, 138B, 138C, 138D, collectively tensioner guides 138, and cords 140A, 140B, 140C, 140D, collectively cords 140, respectively.

Tensioner guides 138 are hollow tubular members that are fixed in position relative to outer tube 124. In one example, tensioner guides 138 are fixed to (e.g., mounted by gluing) or integral with outer tube 124. Tensioner guides 138 include cord lumens through which cords 140 extend.

Cords 140, e.g., cables, wires, or other structures capable of being pulled through tensioner guides 138, extend through tensioner guides 138. Cords 140 exit distal ends 139 of tensioner guides 138 and extend to a tensioner ring 142 of positioning mechanism 134. Distal ends 141 of cords 140 are attached to tensioner ring 142.

Tensioner ring 142 is mounted to outer tube 124 adjacent spindle 126. Accordingly, distal ends 141 are attached to outer tube 124 by tensioner ring 142. Although use of tensioner ring 142 is set forth, in another example, distal ends 141 are directly attached to outer tube 124, e.g., using adhesive and/or mechanical fastening means, without use of tensioner ring 142.

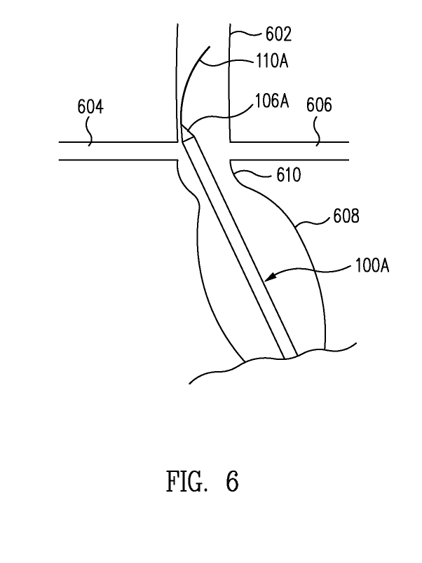

FIG. 4 is a partial cross-sectional view of positionable stent-graft delivery system 100 of FIG. 1 after positioning. Referring now to FIGS. 1 and 4 together, cord 140A, e.g., a first cord, is retracted (tensioned) in the direction of arrow 150 of FIG. 1 through tensioner guide 138A, e.g., by the physician. Retraction of cord 140A, in turn, pulls tensioner ring 142 and thus distal end 128 of outer tube 124, spindle 126, tapered tip 106, and proximal anchor stent ring 306 towards distal end 139 of tensioner guide 138A, e.g., a first tensioner guide. As a result, outer tube 124 is bent (curved) in the radial direction of tensioner 136A. In this manner, stent-graft 302 is readily repositioned as discussed in greater detail below with reference to FIGS. 6, 7, 8, and 9.

After stent-graft 302 is properly position, proximal anchor stent ring 306 is released thus deploying and securing the proximal portion of the stent-graft 302 in position within the vessel as discussed in greater detail below. More particularly, tapered tip 106 is advanced relative to spindle 126 to expose the proximal end of proximal anchor stent ring 306. Upon being released from sleeve 122 of tapered tip 106, the crowns at the proximal end of proximal anchor stent ring 306 self-expand toward and into contact with the wall of the vessel in which stent-graft 302 is being deployed. After deployment and anchoring of proximal anchor stent ring 306 to the vessel wall, primary sheath 102 can be fully retracted to fully deploy the rest of stent-graft 302.

However, in another example, primary sheath 102 is fully retracted prior to release of proximal anchor stent ring 306. To illustrate, instead of being partially retracted at the stage of deployment illustrated in FIGS. 1, 3 and 4, primary sheath 102 is fully retracted while the crowns at the proximal end of proximal anchor stent ring 306 are still radially constrained.

Further, while stent-graft 302 is described above is a self-expanding stent graft. In accordance with another embodiment, instead of being a self-expanding stent-graft, positionable stent-graft delivery system 100 may include an expansion member, e.g., a balloon, which is pressurized to expand and deploy a balloon expandable stent-graft.

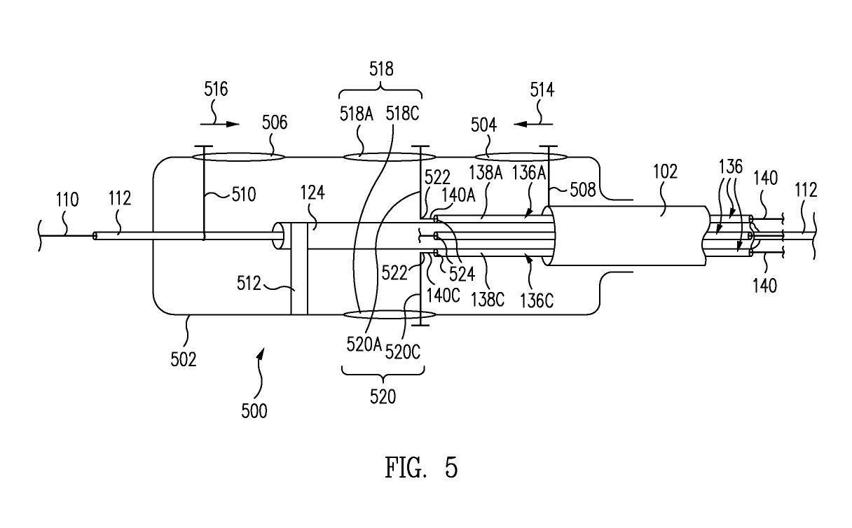

FIG. 5 is a handle 500 of positionable stent-graft delivery system 100 of FIG. 1. Handle 500 includes a housing 502 having a primary sheath retraction slot 504 and an inner tube advancement slot 506. A primary sheath actuation member 508, sometimes called a thumb slider, extends from primary sheath 102 and through primary sheath retraction slot 504. Similarly, an inner tube actuation member 510, sometimes called a thumb slider, extends from inner tube 112 and through inner tube advancement slot 506. Further, outer tube 124 is mounted to housing 502 by an outer tube support 512.

To retract primary sheath 102 relative to outer tube 124, primary sheath actuation member 508 is moved (retracted), e.g., by the physician, in the direction of arrow 514. To advance inner tube 112 relative to outer tube 124, inner tube actuation member 510 is moved (advanced), e.g., by the physician, in the direction of arrow 516.

Further, housing 502 includes a plurality of cord retraction slots 518 corresponding to tensioners 136. Cord actuation members 520, sometimes called thumb sliders, extend from proximal ends 522 of cords 140 and through cord retraction slots 518. Cords 140 extend distally from cord actuation members 520 and into tensioner guides 138 through proximal ends 524 of tensioner guides 138.

In accordance with this example, a cord actuation member 520 is attached to each respective cord 140 and extends through a respective cord retraction slot 518. Thus, in accordance with this example, there are four cord actuation members 520 and four cord retraction slots 518, although only two of each are illustrated in the view of FIG. 5.

More particularly, cord actuation members 520A, 520C are attached to cords 140A, 140C and extend through cord retraction slots 518A, 518C, respectively.

To retract a cord 140 through a respective tensioner guide 138 and relative to outer tube 124, the respective cord actuation member 520 is moved (retracted), e.g., by the physician. This causes the distal end 128 of outer tube 124 to be bent (curved) in the direction of the tensioner 136 as discussed above. As each cord 140 is radially oriented 90 degrees from the adjacent cord 140, i.e., a cord 140 is radially located at the 0, 90, 180 and 270 degree positions, outer tube 124 can be bent in any one of the four directions by actuating the respective cord actuation member 520.

Although one example of a handle is set forth in FIG. 5, in light of this disclosure, those of skill in the art will understand that other handles can be used. Illustratively, handles having ratcheting mechanisms, threaded mechanisms, or other mechanisms to retract the primary sheath, advance the inner tube, and retract the cords relative to the outer tube are used.

Further, to increase the number of directions in which the outer tube can be bent, a positionable stent-graft delivery system similar to positionable stent-graft delivery system 100 is formed with more than four tensioners and associated cord actuation members and cord retraction slots. In yet another example, a positionable stent-graft delivery system similar to positionable stent-graft delivery system 100 is formed with only one, two, or three tensioners, i.e., less than four tensioners, and associated cord actuation members and cord retraction slots. Cord actuation member may be mounted or connected to a manipulation ring (not shown) where each end of the cord manipulated is connected to a circumferential ring to make it easy to pull on two (or more when more than four cords are used) simultaneously with different forces to make the tip easy to manipulate in any lateral direction.

FIG. 6 is a partial cross-sectional view of a positionable stent-graft delivery system 100A located within a diseased vessel 602, e.g., the abdominal aorta, in accordance with one embodiment. Referring now to FIG. 6, a pair of branch vessels 604, 606, e.g., the renal arteries, branch from diseased vessel 602. Further, diseased vessel 602 includes an aneurysm 608 formed therein. Aneurysm 608 includes a neck 610 (sealing surface all the way around a vessel) extending between branch vessels 604, 606 and aneurysm 608.

Diseased vessel 602 is an example of what is considered a complex anatomy. More particularly, instead of extending straight down from branch vessels 604, 606 where the edge of the neck would be substantially perpendicular to a longitudinal axis of the vessel as the vessel approaches from below (as seen here), diseased vessel 602 dramatically curves from branch vessels 604, 606 such that the neck edge is not perpendicular to the vessel axis as it approaches from below.

To deliver positionable stent-graft delivery system 100A within diseased vessel 602, a guide wire 110A is initially passed through diseased vessel 602. Positionable stent-graft delivery system 100A is advanced over guide wire 110A such that a tapered tip 106A of positionable stent-graft delivery system 100A is located near branch vessels 604, 606 as illustrated in FIG. 6.

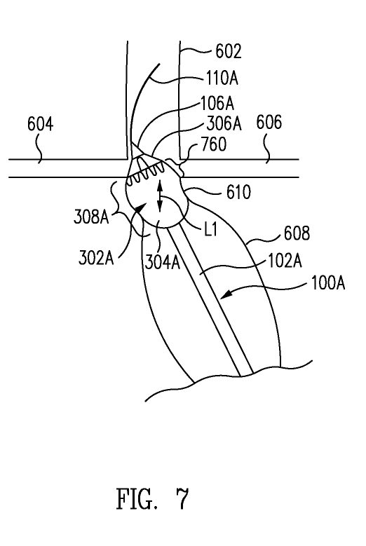

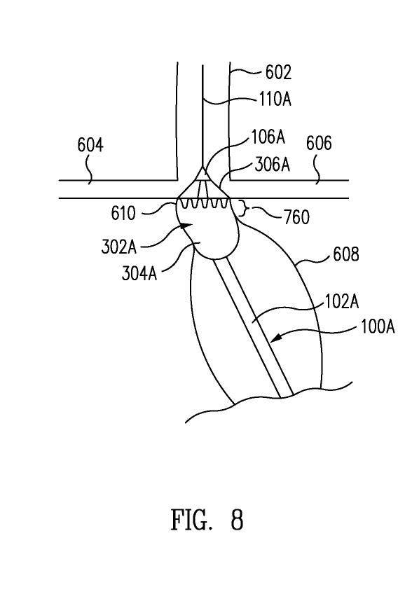

FIG. 7 is a partial cross-sectional view of positionable stent-graft delivery system 100A within diseased vessel 602 of FIG. 6 at a later stage during deployment of a stent-graft 302A of positionable stent-graft delivery system 100A. Referring now to FIG. 7, a primary sheath 102A is partially retracted such that a proximal portion 308A of stent-graft 302A is exposed. Upon being exposed, proximal portion 308A self-expands while the proximal portion (crowns) of a proximal anchor stent ring 306A is radially constrained within tapered tip 106A.

As illustrated in FIG. 7, stent-graft 302A end is deployed at an angle that is not perpendicular to a longitudinal axis L1 of neck 610 of aneurysm 608. More particularly, a portion 760 of a graft material 304A back from the proximal edge of the graft material of stent-graft 302A extends beyond neck 610 thus partially blocking branch vessel 606.

FIG. 8 is a partial cross-sectional view of positionable stent-graft delivery system 100A within diseased vessel 602 of FIG. 7 at a later stage during deployment of stent-graft 302A. Referring now to FIG. 8, stent-graft 302A is repositioned by the physician retracting a cord in a manner similar to that discussed above. Accordingly, portion 760 of graft material 304A is retracted and repositioned such that portion 760 is moved into contact with neck 610, instead of extending beyond neck 610, while the opposite side of the stent graft remains or is repositioned to maintain it contact with the neck 610.

Placing the proximal end of graft material 304A of stent-graft 302A at the top of neck 610 results in a maximum sealing area between graft material 304A and neck 610 (the area of contact between graft material 304A and neck 610). Accordingly, the effectiveness of the seal between graft material 304A and neck 610 of aneurysm 608 is maximized.

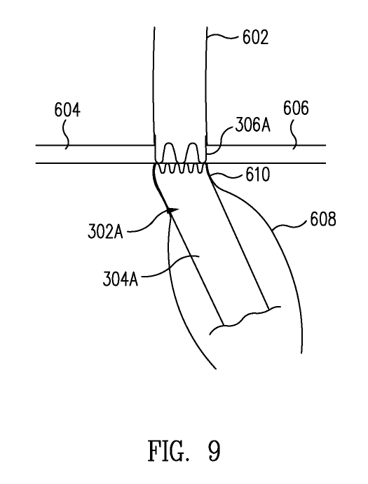

FIG. 9 is a partial cross-sectional view of positionable stent-graft delivery system 100A within diseased vessel 602 of FIG. 8 at a with the proximal end having been deployed. Referring now to FIG. 9, proximal crowns of anchor stent ring 306A are released from tapered tip 106A and deployed, e.g., by advancing tapered tip 106A, as discussed above. Further, primary sheath 102A is fully retracted thus completely deploying graft material 304A and thus the proximal end if not all of stent-graft 302A. In other examples, primary sheath 102A is deployed prior to or simultaneously with proximal anchor stent ring 306A.

A method of deploying a stent-graft with a positionable stent-graft delivery system includes the steps of: radially constraining a proximal anchor stent ring of the stent-graft with a tip capture mechanism of the positionable stent-graft delivery system; radially constraining a graft material of the stent-graft with a primary sheath of the positionable stent-graft delivery system; partially retracting the primary sheath to expose a portion of the stent-graft; and retracting a cord through a tensioner guide of the positionable stent-graft delivery system to move the tip capture mechanism and reposition the proximal anchor stent ring and may further comprise releasing the proximal anchor stent ring from the tip capture mechanism.

The drawings and the forgoing description gave examples according to the present invention. Numerous variations, whether explicitly given in the specification or not, such as differences in structure, dimension, and use of material, are possible.

What is claimed is:

1. A positionable stent-graft delivery system comprising:
a stent-graft including a proximal portion of said stent-graft;
a positioning mechanism for positioning said proximal portion of said stent graft comprising tensioners, said tensioners comprising:
tensioner guides; and
cords extending through said tensioner guides, said cords capable of being moved through said tensioner guides.

2. The positionable stent-graft delivery system of claim 1 further comprising:
a tip capture mechanism radially constraining said proximal portion of said stent-graft;
wherein said positioning mechanism for positioning said proximal portion of said stent graft also positions said tip capture mechanism.

3. The positionable stent-graft delivery system of claim 2 further comprising an outer tube, said tip capture mechanism further comprising a spindle mounted on a distal end of said outer tube.

4. The positionable stent-graft delivery system of claim 3 wherein distal ends of said cords are attached to said outer tube adjacent said spindle.

5. The positionable stent-graft delivery system of claim 4 wherein said positioning mechanism further comprises a tensioner ring mounted to said outer tube adjacent said spindle, said distal ends of said cords being attached to said tensioner ring and thereby to said outer tube.

6. The positionable stent-graft delivery system of claim 4 wherein said tensioner guides are fixed in position relative to said outer tube.

7. The positionable stent-graft delivery system of claim 6 wherein said tensioner guides comprise cord lumens through which said cords extend, said cords exiting distal ends of said tensioner guides.

8. The positionable stent-graft delivery system of claim 6 wherein said cords comprise a first cord and said tensioner guides comprise a first tensioner guide comprising a distal end, wherein retraction of said first cord through said first tensioner guide moves said tip capture mechanism towards said distal end of said first tensioner guide.

9. The positionable stent-graft delivery system of claim 8 wherein retraction of said first cord through said first tensioner guide bends said outer tube in a direction of said first tensioner guide.

10. The positionable stent-graft delivery system of claim 4 wherein said tensioner guides are fixed to said outer tube.

11. The positionable stent-graft delivery system of claim 1 wherein said tensioners are radially spaced equally from one another.

12. The positionable stent-graft delivery system of claim 11 wherein said positioning mechanism comprises four of said tensioners, each tensioner being radially oriented 90 degrees from an immediately adjacent tensioner.

13. The positionable stent-graft delivery system of claim 11 wherein said positioning mechanism comprises four of said tensioners comprising a first tensioner, a second tensioner, and a third tensioner, said first tensioner being radially oriented 90 degrees from said second tensioner and in an opposite direction being radially oriented 90 degrees from said third tensioner.

14. The positionable stent-graft delivery system of claim 2 further comprising an inner tube, said tip capture mechanism comprising a tapered tip mounted on a distal end of said inner tube.

15. The positionable stent-graft delivery system of claim 14 further comprising an outer tube, said tip capture mechanism further comprising a spindle mounted on a distal end of said outer tube, said proximal portion of said stent-graft being radially constrained in an annular space between a spindle body of said spindle and a sleeve of said tapered tip.

16. The positionable stent-graft delivery system of claim 15 wherein said spindle further comprises spindle pins, said stent-graft extending around said spindle pins.

17. The positionable stent-graft delivery system of claim 15 wherein said inner tube extends through said outer tube, said inner tube capable of being moved relative to said outer tube to release said proximal portion of said stent-graft from said tip capture mechanism.

18. The positionable stent-graft delivery system of claim 1 further comprising a primary sheath, wherein said stent-graft comprises:
a proximal anchor stent ring, wherein a proximal portion of said proximal anchor stent ring is radially constrained by said tip capture mechanism; and
a graft material attached to said proximal anchor stent ring, wherein said graft material is radially constrained by said primary sheath.

19. A positionable stent-graft delivery system comprising:
a stent-graft;
an inner tube comprising a tip mounted on a distal end of said inner tube;
an outer tube comprising a spindle mounted on a distal end of said outer tube, a proximal anchor stent ring of said stent-graft being radially constrained in an annular space between a spindle body of said spindle and a sleeve of said tapered tip;
a primary sheath radially constraining a graft material of said stent-graft;
a positioning mechanism for positioning said stent-graft comprising:
tensioner guides; and
cords extending through said tensioner guides; and
a handle comprising:
a primary sheath actuation member for retracting said primary sheath relative to said outer tube;
an inner tube actuation member for advancing said inner tube relative to said outer tube; and
cord actuation members for retracting said cords through said tensioner guides.

20. A method of deploying a stent-graft with a positionable stent-graft delivery system comprising:
radially constraining a proximal anchor stent ring of said stent-graft with a tip capture mechanism of said positionable stent-graft delivery system;
radially constraining a graft material of said stent-graft with a primary sheath of said positionable stent-graft delivery system;
partially retracting said primary sheath to expose a portion of said stent-graft; and
retracting a cord through a tensioner guide of said positionable stent-graft delivery system to move said tip capture mechanism and reposition said proximal anchor stent ring.

21. The method of claim 20 further comprising releasing said proximal anchor stent ring from said tip capture mechanism.

22. The method of claim 20 further comprising:
wherein said partially retracting said primary sheath comprises positioning a portion of a graft material of said stent-graft beyond said neck, and
wherein said retracting a cord through a tensioner guide comprises repositioning said portion of said graft material into contact with said neck.
